(19) United States
(12) Patent Application Publication
(10) Pub. No.: US 2026/0334856 A1
(43) Pub. Date: Oct. 29, 2026

(54) WEARABLE SYSTEM FOR MONITORING AND ENHANCING ADHERENCE TO REHABILITATION EXERCISES

(71) Applicant: KNEE2ME INC., Burlington (CA)

(72) Inventors: Oluwatoni BODUNRIN, Burlington (CA); Rene GILLILAND-ROCQUE, Oakville (CA); Madison FERNANDEZ, North York (CA)

(73) Assignee: KNEE2ME INC., Burlington (CA)

(21) Appl. No.: 19/539,631

(22) Filed: Feb. 13, 2026

(30) Foreign Application Priority Data
Apr. 28, 2025 (CA) 3,271,984

(51) Int. Cl. A61B 5/00; A61B 5/11

(52) U.S. Cl. CPC A61B 5/4528; A61B 5/1121; A61B 5/4836; A61B 5/6802; A61B 5/6831; A61B 5/742; A61B 2505/09; A61B 2562/0219

(57) ABSTRACT

A wearable system and method for monitoring and guiding rehabilitation exercises is disclosed, particularly for knee physiotherapy. The system includes a wearable sensor module (comprising an inertial measurement unit, range-of-motion sensor, onboard processor, and wireless transceiver) and a companion software application on an external device. The sensor module computes joint-angle trajectories and exercise-performance metrics in real time, transmitting data to the software application, which provides immediate visual and/or auditory feedback to guide correct movement. The system records exercise repetitions with time-stamped adherence data and updates configuration settings based on user performance trends. Additional features include a magnetometer for enhanced accuracy, adaptive filtering algorithms, and an LED array for direct visual feedback. The method enables unsupervised physiotherapy by comparing real-time metrics to target trajectories and issuing corrective prompts. A gamified application tracks progress, shares metrics with accountability partners, and integrates with electronic health records, enhancing adherence and recovery outcomes.

### FIELD OF INVENTION

This application claims priority to Canadian Patent Application No. 3,271,984 filed Apr. 28, 2025, the entirety of which is included herein by reference.

The present disclosure pertains to medical rehabilitation technologies, specifically wearable systems designed for monitoring and guiding physiotherapy exercises to improve adherence and recovery outcomes.

### BACKGROUND

The field of medical rehabilitation has seen growing interest in wearable technologies that support physiotherapy exercises aimed at restoring musculoskeletal function.

Rehabilitation protocols rely on precise execution of prescribed exercises, consistent adherence to daily regimens, and timely feedback to correct movement patterns. Clinicians prescribe tailored exercise routines based on individual injury profiles, aiming to optimize joint mobility, muscle strength, and neuromuscular control. Effective at-home systems are required to not only record exercise repetitions but also guide patients through correct movement trajectories, offer motivational cues, and maintain comprehensive logs of performance trends. Achieving these goals demands seamless integration of hardware, software, and user interfaces designed with both clinical accuracy and patient usability in mind.

Despite advances in sensor miniaturization and mobile application development, many existing solutions fall short of delivering a fully integrated, patient-centric rehabilitation platform. Systems currently available often require specialized setup in a clinical environment, limiting their suitability for independent use during home-based therapy. Moreover, feedback mechanisms tend to be rudimentary or delayed, relying on post-session data reviews rather than instantaneous guidance. As a result, patients may inadvertently perform exercises with suboptimal form, undermining therapeutic efficacy and prolonging recovery timelines.

A particularly pressing challenge lies in the absence of continuous, real-time oversight when patients undertake rehabilitation exercises outside supervised settings. Without immediate corrective prompts or visual cues, deviations from target movement paths can go unnoticed, increasing the risk of re-injury or compensatory loading patterns. Furthermore, the lack of automated adherence tracking and contextual guidance diminishes patient motivation, leading to missed sessions and inconsistent effort. Addressing this specific deficiency is critical to ensuring that home-based rehabilitation mirrors the fidelity and responsiveness of in-clinic therapy, enhancing outcomes and reducing long-term healthcare burdens.

### SUMMARY

In one embodiment, a rehabilitation monitoring system comprises a wearable sensor module configured to be secured around a user's body part by for example an adjustable elastic strap with a breathable skin-facing surface for extended wear without irritation or other suitable attachment means and a companion software application on an external electronic device. The wearable sensor module has an inertial measurement unit having a plurality of range-of-motion sensors including a gyroscope and an accelerometer that jointly generate raw motion signals to capture angular displacement of a joint, an onboard processor that in real time computes joint-angle trajectories and exercise-performance metrics from those signals, and a wireless transceiver operable to transmit the computed metrics to the external electronic device and to receive configuration data from the external electronic device while the user performs rehabilitation exercises.

The companion software application is configured to receive the transmitted exercise-performance metrics, generate simultaneous visual and/or auditory feedback guiding the user toward correct movement in real time. The companion software application may further record each completed exercise repetition with an adherence timestamp and transmit updated configuration data back to the wearable sensor module based on detected user-performance trends.

Both the wearable sensor module and the companion software application communicate bidirectionally during the rehabilitation exercises. This enables the system to generate simultaneous visual and/or auditory feedback that guides the user toward correct movement in real time; and transmit the configuration data back to the wearable sensor module in accordance with detected user-performance trends.

In some aspects, the wearable sensor module further incorporates a selectively activatable magnetometer whose output is combined with inertial measurement unit data to enhance joint-angle accuracy when magnetic disturbance is below a stored threshold. A power supply for the wearable sensor module is provided which may consist of a non-rechargeable battery such as a standard AAA battery or a rechargeable battery with an integrated USB-C charging port and LED charge/status indicator. In other aspects, the onboard processor may apply at least one of Savitzky-Golay filtering, adaptive moving-average filtering, or Kalman filtering to the raw motion signals, detects peak range-of-motion values for each repetition, and transmits only those peak values along with exercise-type metadata.

In further aspects, the module includes an outward-facing LED array controlled by the onboard processor to emit color-coded light patterns indicative of current joint-angle magnitude so the user can receive direct visual feedback without referencing the external device . There may be a second a single colour LED that flashes patterns to indicate whether the device is paired via Bluetooth to another device.

In another embodiment, a computer-implemented method guides a user through a physiotherapy session without clinical supervision by securing the wearable sensor module to the user's body, calculating joint-angle trajectories and exercise-performance metrics in real time on the module, wirelessly transmitting those metrics to an external computing device running the companion application, comparing the real-time metrics to a stored target trajectory, and outputting immediate corrective prompts—selected from sound, vibration, or on-screen highlights—whenever the user deviates from the target. Upon completion of each exercise set, the method may further record a time-stamped entry that includes repetition count, maximum flexion and extension angle reached, and a movement-quality score, updates a streak counter representing consecutive days of exercise completion, automatically synchronizes the entry to a cloud account accessible by an authorized accountability partner when network connectivity is available (and stores the entry locally for later synchronization when connectivity is absent), and issues a reminder notification to the mobile device reminders to the user and accountability partner if a scheduled session is missed.

In a further embodiment, a gamified rehabilitation software application executable on a portable electronic device is configured to receive exercise-performance metrics from the wearable sensor module via a wireless interface, maintain the consecutive-day streak counter, share aggregate performance metrics with at least one designated accountability partner over a network connection, unlock guided exercise videos, and visually represent user progress on an interactive chart updated after each session. The application is also configured to export stored performance metrics as human-readable PDF reports and as machine-readable FHIR-compliant records for clinician review and integration into external electronic health-record systems.

These and other aspects of the system will be more clearly understood from the following detailed description taken in conjunction with the accompanying drawings and claims.

Further features of the invention will be described or will become apparent in the course of the following detailed description.

### BRIEF DESCRIPTION OF DRAWINGS

In order that the invention may be more clearly understood, an embodiment will now be described in detail by way of example, with reference to the accompanying drawings, in which:

FIG. 1 is a perspective front view of one embodiment of a wearable sensor module according to the present disclosure.

FIG. 2. is a perspective front view of the wearable sensor module of FIG. 1 on a patient's lower leg adjacent the knee with a second sensor above the knee and connected to the wearable sensor module.

FIG. 3 is a perspective side view of the wearable sensor module of FIG. 2 on a patient's leg below the knee.

FIG. 4 is a perspective front view of the wearable sensor module of FIGS. 2 and 3 on a patient's leg below the knee during an exercise with the leg extended.

FIG. 5 is a perspective front view of the wearable sensor module of FIG. 4 during an exercise with the leg at rest with the foot on the floor.

FIG. 6 illustrates one embodiment of the hardware architecture diagram for a wearable system designed for monitoring and guiding physiotherapy exercises according to the present disclosure.

FIG. 7 is an exploded view of the parts of the wearable sensor module without the strap of FIG. 1.

FIG. 8. Is an exploded view of the assembled wearable sensor module of FIG. 7.

FIG. 9 is a circuit diagram for the PCB of the wearable sensor module of FIG. 7.

FIG. 10 is a plan drawing of the PCB of the wearable sensor module of FIG. 7 showing layout of LED arrays, onboard processor in the form of a microcontroller unit, inertial measurement unit, power button and wireless transceiver with a Bluetooth module

FIG. 11 is a Flow Chart for one embodiment of the companion software for a system for monitoring and guiding rehabilitation exercises according to the present disclosure.

FIG. 12 is a User Flow Chart for one embodiment of a system for monitoring and guiding rehabilitation exercises according to the present disclosure.

Similar references are used in different figures to denote similar components

### DETAILED DESCRIPTION

Aspects of the disclosure are described more fully hereinafter with reference to the accompanying drawings, which form a part hereof, and which show, by way of illustration, example features. The features can, however, be embodied in many different forms and should not be construed as limited to the combinations set forth herein; rather, these combinations are provided so that this disclosure will be thorough and complete and will fully convey the scope. The following detailed description is, therefore, not to be taken in a limiting sense.

As used in the disclosure and accompanying drawings, BLE stands for Bluetooth Low Energy. It's a wireless communication technology designed for low-power applications and is often referred to as Bluetooth Smart. BLE is a subset of the Bluetooth technology standard, offering reduced power consumption compared to traditional Bluetooth. ROM means range of motion. Inertial Measurement Unit (IMU) is an electronic device that measures and reports a body's specific force, angular rate, and sometimes the orientation of the body, using a combination of accelerometers, gyroscopes, and sometimes magnetometers. When the magnetometer is included, IMUs are referred to as IMMUs.

The present system addresses the challenges noted above by introducing a novel method designed to facilitate independent monitoring and guidance of rehabilitation exercises. The system comprises a wearable sensor module configured to be worn adjacent a joint being rehabilitated, having an inertial measurement unit comprising a range-of-motion sensors. In the embodiments illustrated the rehabilitation is directed to the knee joint. The system can be used for other joints like hip, elbow, shoulder, neck, wrist or ankle with adjustments to the hardware attachment and content and calibration of the companion software app. These sensors capture detailed data on joint movement, which is processed in real time by an onboard processing unit. The processed data is then transmitted wirelessly to an external electronic device, where a companion software application provides the user with real-time visual and auditory feedback on the wearable sensor module, tracks exercise performance, and generates notifications to encourage adherence to prescribed rehabilitation exercises. This approach empowers patients to actively participate in their recovery process, offering a user-friendly solution that bridges the gap between clinical recommendations and at-home exercise execution.

The following detailed description provides illustrative embodiments of the described technology, which pertains to the field of medical rehabilitation technologies, specifically wearable systems designed to monitor and guide physiotherapy exercises. The described technology addresses challenges in adherence, feedback, and performance tracking during rehabilitation, particularly for musculoskeletal recovery. While the embodiments focus on knee rehabilitation, the principles and features described herein may be adapted for other joints, such as the hip, elbow, shoulder, wrist, or ankle, with appropriate modifications to hardware and software configurations. Variations, rearrangements, and modifications of the described components, methods, or systems—whether explicitly mentioned or not—are considered within the scope of the described subject matter, as long as they align with the principles and objectives disclosed herein.

The disclosed wearable system and method address the significant challenge of ensuring consistent adherence to prescribed physiotherapy exercises, particularly during unsupervised, at-home rehabilitation. By providing real-time feedback, detailed performance tracking, and motivational tools, the disclosed technology bridges the gap between clinical oversight and independent exercise execution, reducing the risk of improper form, prolonged recovery, and re-injury.

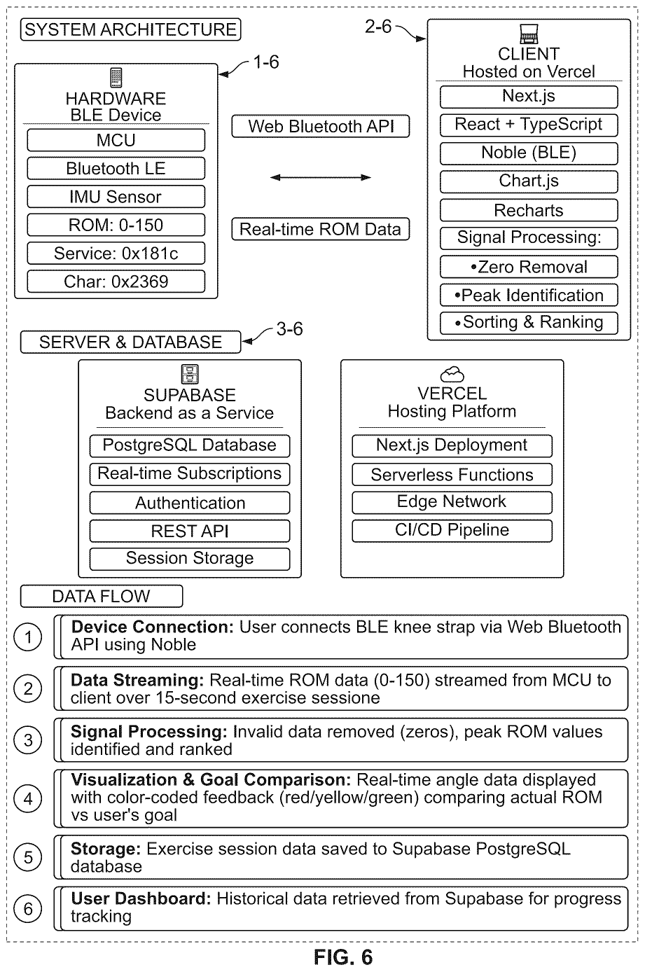

The system is employed in the context of musculoskeletal rehabilitation, with the disclosed embodiment focused on knee recovery, though it is adaptable for other joints such as the hip, shoulder, or ankle. Referring to FIG. 6, the hardware architecture diagram for one embodiment of the wearable system for monitoring and guiding rehabilitation exercises according to the present invention is illustrated. A wearable sensor module, generally indicated at 1-6, is secured adjacent the joint to be rehabilitated, operates under typical conditions of ambient temperature and is designed for extended daily use. The wearable sensor module 1-6 is configured to capture joint movement data using an inertial measurement unit having range-of-motion sensors, processes the data in real time, and transmits the information wirelessly, using Bluetooth technology in the embodiment illustrated, to a companion software application on a user's electronic device 2-6 such as a desktop computer, laptop, tablet or mobile device. The companion software application, which is a part of the system, is designed to enhance the user experience by providing comprehensive support and feedback during rehabilitation exercises. This software is compatible with both mobile and desktop platforms, ensuring accessibility and convenience for users. It offers guided exercise protocols prescribed by physiotherapists and downloaded to the user's electronic device 2-6 from a backend server and database 3-6, complete with instructional video demonstrations to ensure correct exercise execution. The application delivers real-time visual and auditory feedback on movement quality and range of motion, allowing users to make immediate adjustments to their technique. The application provides immediate corrective prompts, tracks adherence through time-stamped logs, and visualizes progress via interactive charts. Representative results demonstrate improved exercise accuracy and adherence rates, with users achieving consistent range-of-motion targets over consecutive sessions. Alternative configurations, such as different sensor placements or communication protocols, and additional features like gamified progress tracking or clinician data sharing, also fall within the scope of the described system, ensuring flexibility for diverse rehabilitation needs.

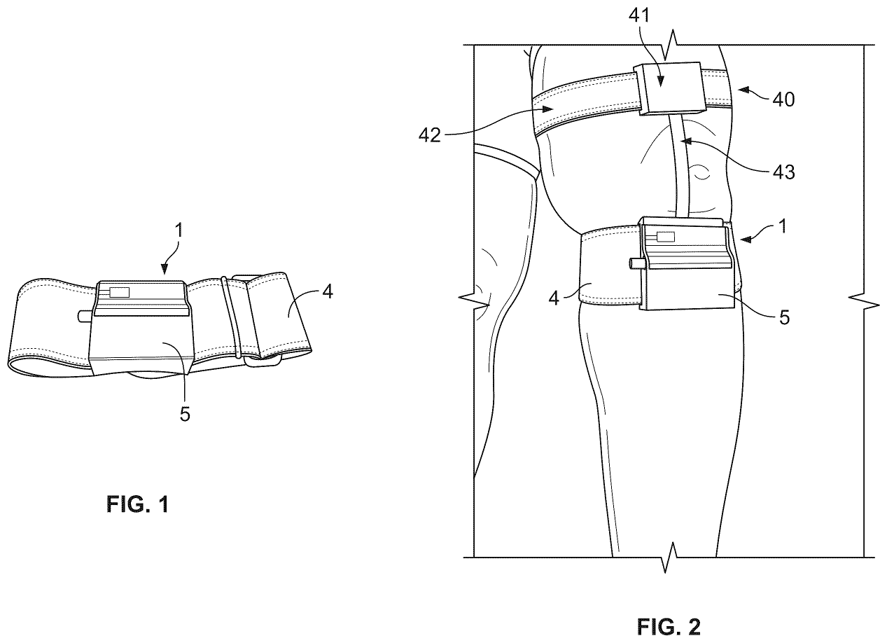

Referring to FIGS. 1-5 and 7-8, in one embodiment, a wearable sensor module generally indicated at 1 in accordance with the present invention is illustrated. It is designed to be worn on a user's body adjacent the joint to be monitored and guided during rehabilitation exercises. The wearable sensor module, generally indicated at 1, is sensor module designed to be worn using an adjustable elastic strap 4. The device is ergonomic and lightweight for extended daily wear. designed with a rigid external housing 5 made from durable materials such as polycarbonate or ABS plastics. Alternatively, the external housing 5 may be constructed from flexible, breathable fabric to enhance comfort during extended wear, particularly for users with sensitive skin.

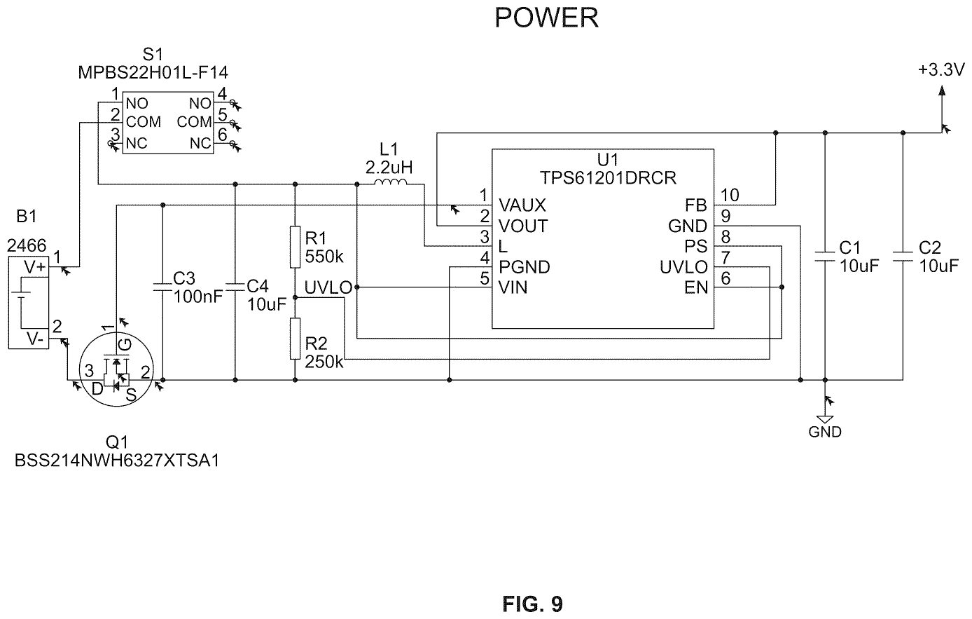

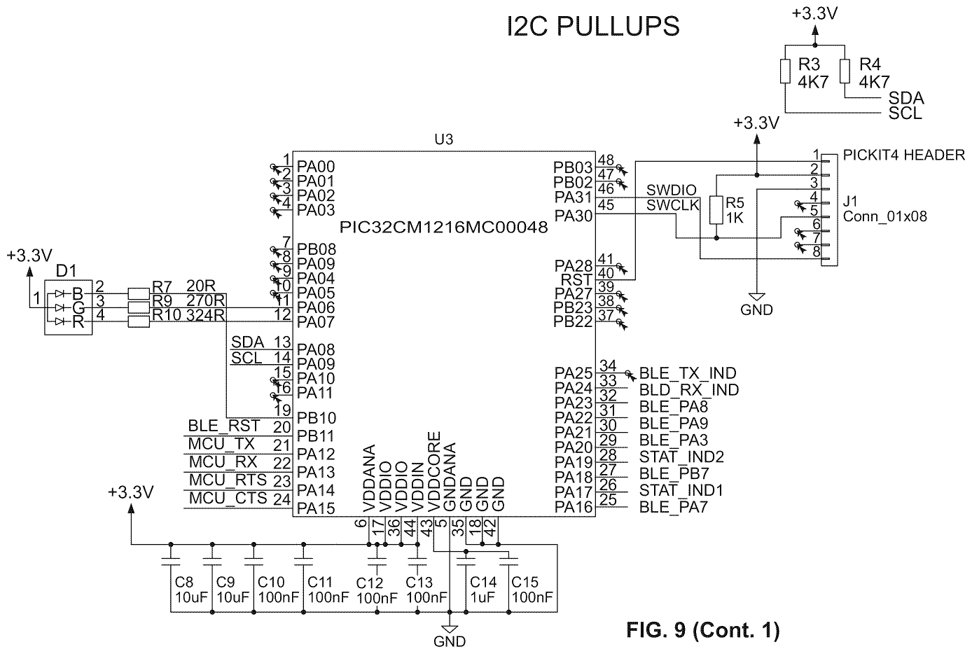

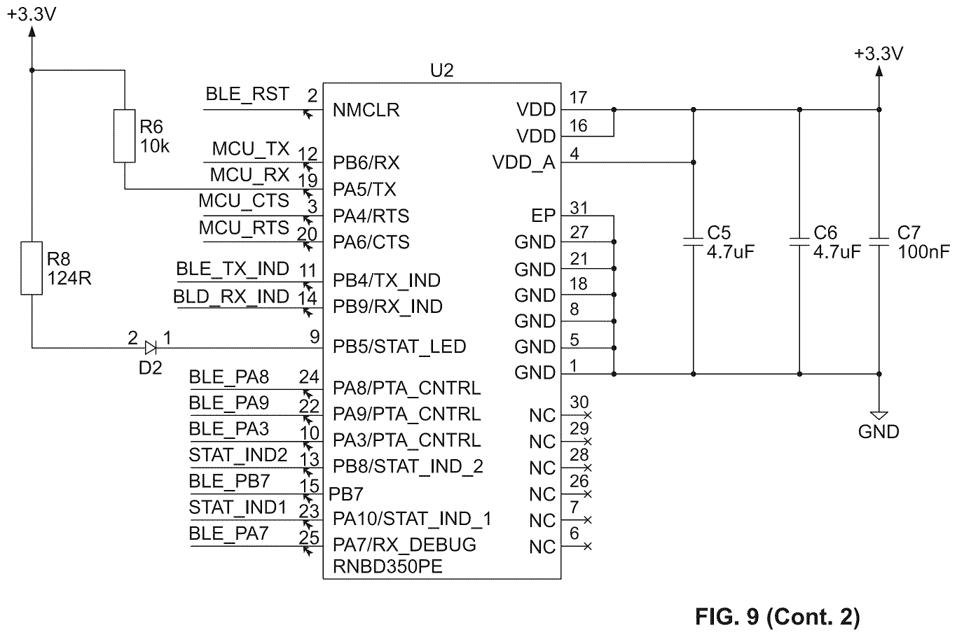

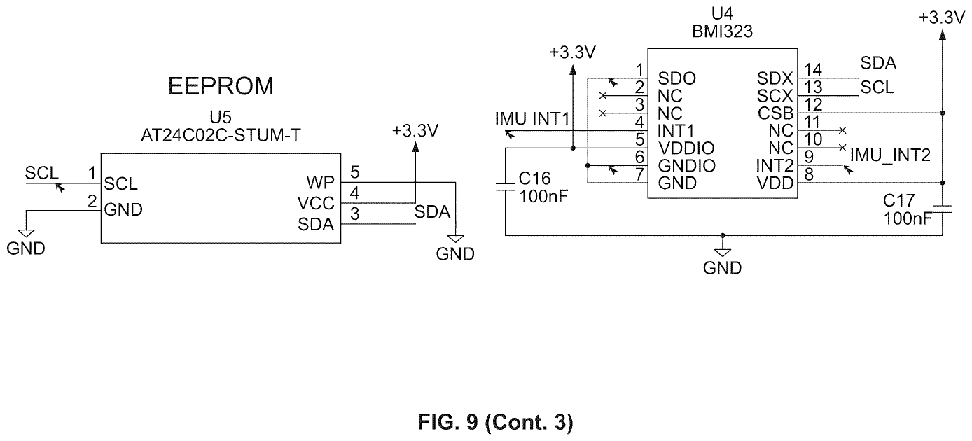

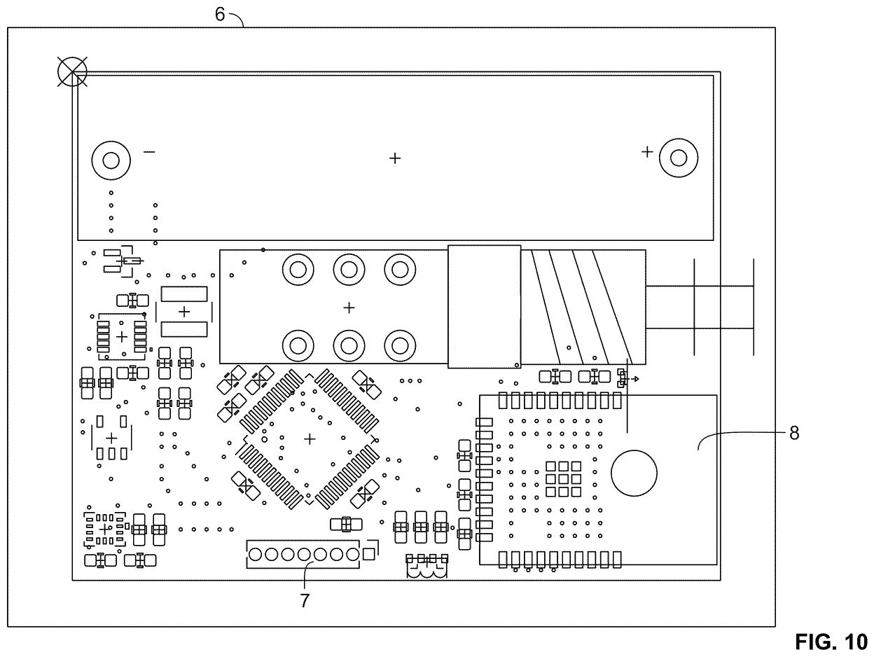

Within the external housing 5 is a printed circuit board (PCB) 6 containing as best illustrated in FIGS. 9 and 10 an inertial measurement unit including a plurality of sensors. The inertial measurement unit in the embodiment illustrated includes range-of-motion sensors in the form of a gyroscope and accelerometer and in some embodiments may include, a magnetometer is selectively activated to improve joint-angle accuracy under high magnetic interference conditions. The range-of-motion sensors in the inertial measurement unit can be implemented using different technologies, such as optical sensors or strain gauges or capacitive sensors, each offering varying levels of precision and adaptability to different exercise protocols.

The onboard processor on the PCB 6 may range from basic microcontrollers for simple data processing to advanced processors capable of executing complex algorithms, such as Kalman filtering or adaptive moving-average filtering, to refine motion signals. A wireless transceiver 8 is provided on the PCB 6 and may utilize Bluetooth Low Energy (BLE) for efficient data transmission, but alternative communication protocols such as Wi-Fi or Zigbee can be employed to meet specific connectivity requirements.

An outward-facing LED array 7 is provided on the PCB 6 that provides real-time visual feedback through color-coded light patterns, allowing users to monitor their progress without relying on the external electronic device. There may be a second a single colour LED, blue in the embodiment illustrated, that flashes patterns to indicate whether the device is paired via Bluetooth to another device.

A second sensor unit, generally indicated at 40, is shown in FIGS. 2-5 connected above the knee using band 42. In the embodiment illustrated inertial measurement unit is provided in second sensor housing 41. In the embodiment illustrated the inertial measurement unit utilizes a BMI323 IMU. It outputs acceleration and angular speed for three axes. The second sensor 40 outputs are used in conjunction with the main sensor module 1 to determine the joint angle. The external sensor provides a reference orientation for the x, y, and z axes from which the range of motion is calculated. The second sensor 40 enables the device to correctly calculate the range of motion of for example the knee joint when the patient is not sitting with their femur (upper leg) parallel to the ground. This enables the device to function for joints or combinations of joints that may not be performed in the exact same position relative to the gravity vector. A cable 43 carries power and signal between the PCBs on each sensor 1, 40 over a standard i2c bus. There are four pins total forming the bus. Two pins are 3.3 v and ground, and two pins are SDA and SCL for serial data and serial clock respectively

The companion software application, executed on a mobile or desktop device, may offer features such as guided exercise videos, real-time feedback through auditory or visual prompts, and progress tracking with graphical summaries. Additionally, the software may include gamification elements, such as streak counters and milestone rewards, to motivate users and enhance adherence to rehabilitation exercises.

The power supply may consist of a standard AAA battery (not shown) as used in the illustrated embodiment or rechargeable lithium-ion battery with a USB-C charging port and LED status indicators, or other form of a non-rechargeable battery for users seeking a maintenance-free option.

The system incorporates signal processing techniques to ensure accurate assessment and feedback during rehabilitation exercises. Raw sensor data captured by the inertial measurement unit and range-of-motion sensor undergoes filtering and analysis to extract meaningful motion patterns and eliminate erroneous readings that may result from sensor initialization, temporary signal loss, or environmental interference.

In one embodiment, the companion software application first performs data validation by removing invalid data points, such as zero values that may occur during sensor initialization or momentary disconnection. This initial filtering step ensures that subsequent analysis is performed only on valid measurement data representing actual joint movement.

The processed data is then analyzed to identify and rank the highest range-of-motion values achieved during the exercise session. The system sorts the valid angle measurements in descending order and identifies the top angular displacement values, representing the patient's peak performance during each exercise cycle. In one embodiment, the system identifies and stores the top ten highest range-of-motion measurements achieved during the session, allowing for detailed analysis of exercise quality and consistency. The difference between the highest measured angle and minimum measured angle is designated as the range of motion achieved for the session and is used for progress tracking and comparison against the user's prescribed rehabilitation goals.

This peak identification approach provides several advantages over traditional repetition counting methods. By capturing and ranking multiple peak values rather than simply counting cycles, the system can assess not only whether exercises were performed, but also the quality and consistency of movement throughout the session. For example, if a user's goal is 90 degrees of flexion, the system can identify whether they achieved this target consistently across multiple repetitions or whether their range of motion varied significantly during the exercise period.

The system may additionally employ temporal filtering to ensure accurate peak identification. By analyzing the time intervals between successive high-angle measurements, the system can distinguish between distinct exercise repetitions and minor fluctuations in joint angle that occur naturally during movement. This temporal analysis may utilize minimum time thresholds between peaks to ensure that each identified peak represents a separate, complete exercise cycle rather than momentary variations within a single repetition.

In alternative embodiments, the system may employ additional signal processing techniques such as Savitzky-Golay filtering, moving average calculations, or other smoothing algorithms to reduce measurement noise while preserving important features of the motion signal. These techniques may apply polynomial regression to successive subsets of adjacent data points or calculate rolling averages to stabilize sensor readings and provide more consistent feedback to the user. The specific filtering parameters may be adjustable based on the type of exercise, the user's baseline range of motion, sensor sampling rate, and the prescribed rehabilitation protocol, allowing for personalized feedback tailored to individual recovery needs

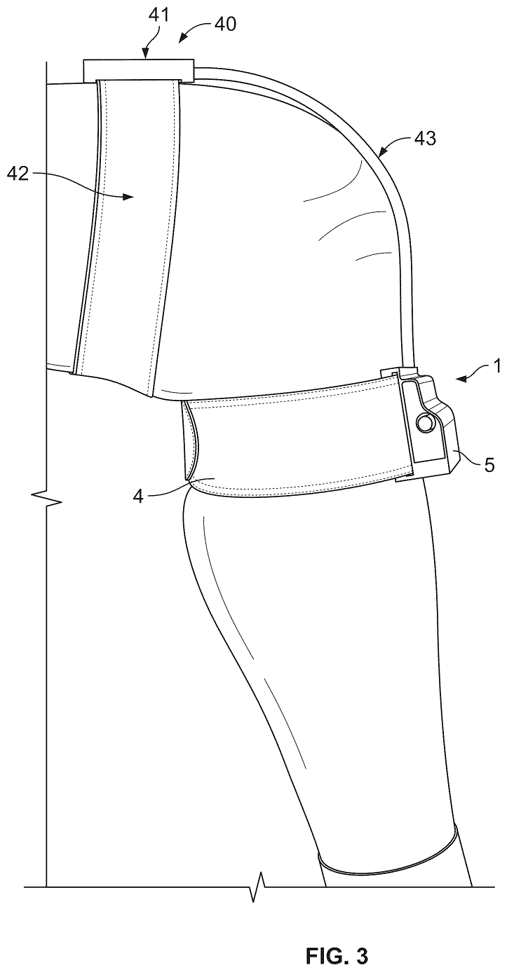

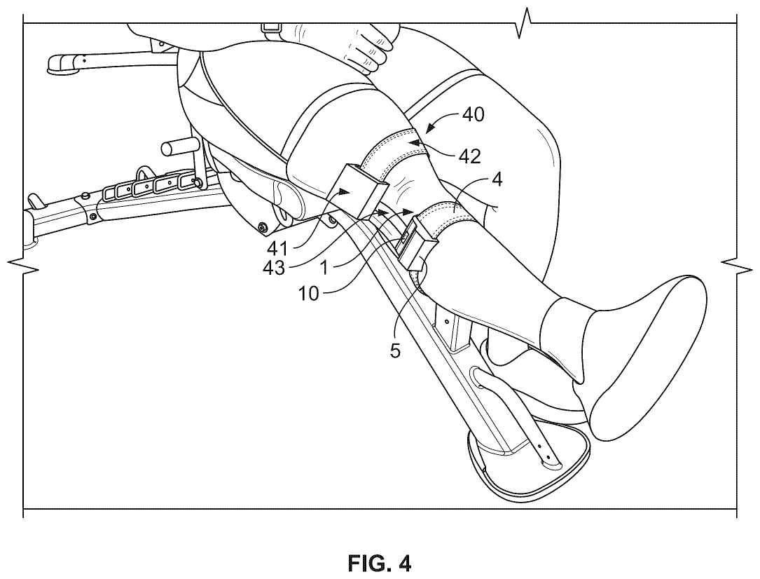

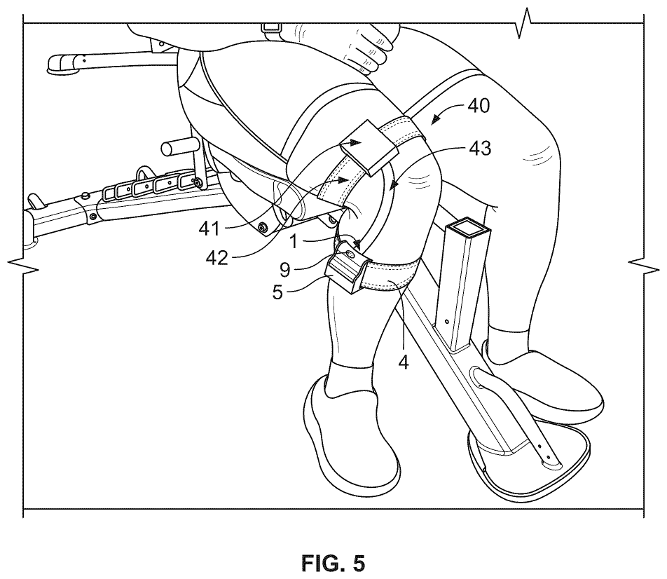

The real time visual and auditory feedback to a patient can be provided by a light indicator as shown in FIGS. 2-5. The LED array 7 on the PCB 6 can light up to provide feedback. Different colored LEDs can indicate if the patient is below target, on target or above target. FIGS. 2-5 show the user in various stages of seated knee extension exercise used to restore full knee extension mobility, particularly following an acute injury or following surgery in the knee, hip or leg. The user has placed the wearable sensor module on their lower leg just below the knee. The wearable sensor module is typically worn forward facing so the user can see the LED array 7 as shown in FIGS. 2 and 3. In FIGS. 4 and 5 the wearable sensor module has been rotated to the outside of the leg so it can be seen better in the Figs. They start by sitting on a chair or bench and extend their knee to the fullest range tolerable and hold a strong isometric contraction for 5-7 seconds. Then they gently lower the leg by bending the knee to complete a repetition. FIG. 5 shows the user's leg bent at 90° with their foot on the floor. This is the starting and ending position for each repetition. The LED array 7 is red and is visible through the sensor external housing at point 9. FIG. 4 shows the user's leg fully extended in the target position and the LED array 7 is green and is visible through the sensor external housing at point 10.

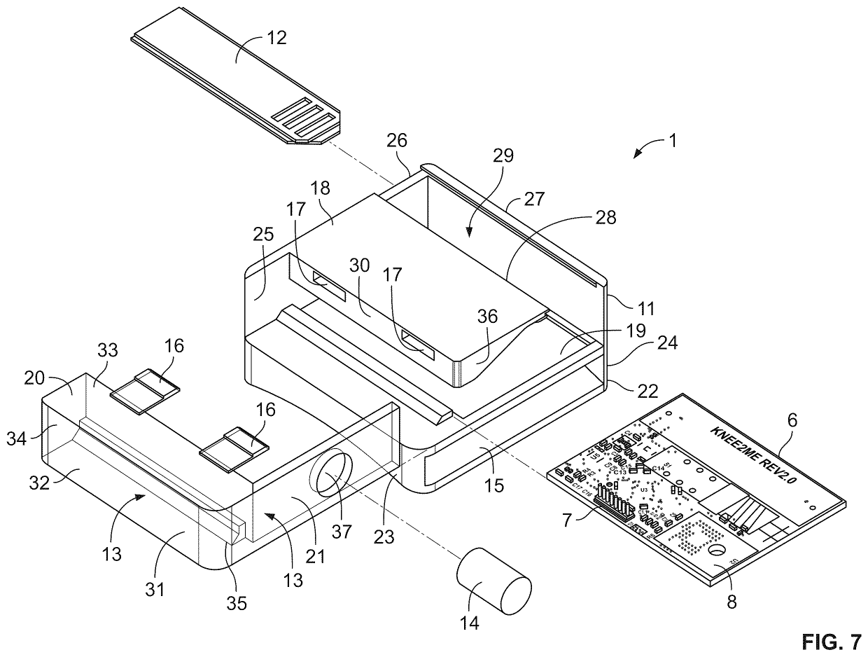

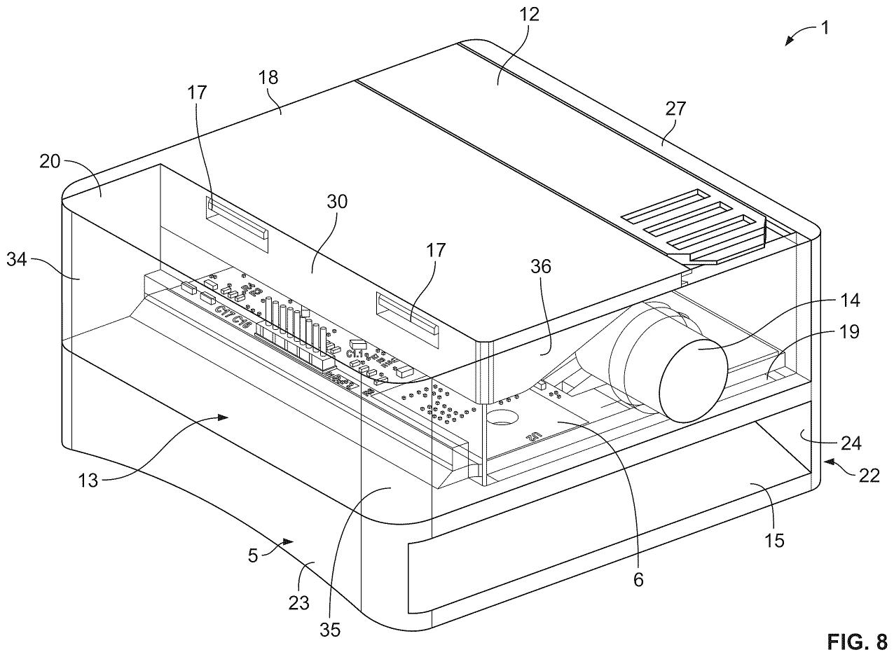

The exploded view in FIG. 7 and the detailed assembled view in FIG. 8 collectively illustrate the structural and functional integration of the wearable sensor module 1. FIG. 7 provides a breakdown of the sensor module's components, the (1) rigid external housing generally indicated at 5 and including the battery cover 12, PCB enclosure 11, retention block 13 and power button 14 and (2) PCB 6. The power supply is not shown.

The PCB enclosure 11 has a generally rectangular configuration with a base section 22 having a slot 15 with end walls 23, 24 for attachment of the elastic band 4 or other suitable connection means. The flat top surface 19 of the base portion 22 is sized and shaped to support the PCB 6 when inserted into the PCB enclosure 11. The bottom end wall 24 extends perpendicularly above top surface 19. The top end wall 23 terminates at top surface 19. A side wall 25 of base section 22 extends perpendicularly above top surface 19 and its top edge 26 is co-planar with the top edge 27 of bottom end wall 24. Extending inwardly from the top edge 26 of side wall 25 is a flange portion 18. The bottom edge 28 of flange portion 18 together with bottom end wall 24 and top surface 19 define a battery compartment 29. The open side of the battery compartment 29 is closed by battery cover 12 designed to slide into slots in the top edge 27 of bottom end wall 24 and flange 18. A pair of locking slots 17 are provided in the top side 30 of flange 18.

The retention block 13 has a rectangular box shaped top body portion 31 with top and bottom walls 32 and 33 and end walls 34,35. End wall 35 extends 21 and is sized and shaped to enclose the free end 36 of flange 18 and the top surface 19 of body portion 22. The front face 20 of retention block is equipped with clips 16 that lock into the slots 17 on flange 18. A hole 37 is provided on extension 21 to permit power button 14 to be inserted into an operable position on PCB 6 when the sensor module is assembled. The rectangular box shaped top body portion 31, top and bottom walls 32 and 33 and end walls 34,35 of retention block 13 is transparent or translucent to enable a user to see the colour of the light from the LED array 7 on PCB 6 when activated to provide real-time visual feedback to the user. A second LED array (not shown) points at the power button 14.

FIG. 8 shows the assembled configuration, highlighting the placement and interaction of these components. Together, these figures demonstrate the modular design and user-centric features of the device, emphasizing the ergonomic and functional attributes of the wearable sensor module 1.

In FIG. 7, the exploded arrangement reveals the internal structure of the external housing 5 of wearable sensor module 1. The battery cover 12 is shown as a detachable component designed to securely enclose the power supply while allowing easy access for maintenance or replacement.

FIG. 8 presents the detailed assembled view of the wearable sensor module 1.

To assemble the wearable sensor module 1, PCB 6 is inserted into the PCB enclosure on the top surface 19 of base section 22. The battery (not shown, is inserted into battery compartment 29 and battery cover 12 inserted into position on the PCB enclosure. Retention block 13 is then slid into position with clips 16 inserted into slots 17 on flange 18 to lock it into position. Power button 14 is then inserted through hole 37 on the extension 21 of retention block 13.

FIG. 9 illustrates the power circuit schematic diagram for the wearable sensor module 1. The primary component in the power management circuit is a TPS61201DRCR integrated circuit U1, which functions as a DC-DC boost converter to regulate the supply voltage. The circuit includes a battery B1, identified as 2466, which serves as the main power source for the module. The power from the battery B1 is managed through a MOSFET switch Q1, BSS214NWH6327XTSA1, and a push-button switch S1, MPBS22010-LF-14, enabling controlled power delivery and user-initiated activation.

The inductor L1, rated at 2.2 μH, and capacitors C1 and C2, each 10 μF, are used for input and output filtering to ensure stable voltage regulation and minimize noise. Additional capacitors C3 (100 nF) and C4 (100 μF) provide further filtering and stabilization within the circuit. The resistors R1 (550 k) and R2 (250 k) are configured to set the feedback and undervoltage lockout thresholds for the boost converter, ensuring reliable operation under varying battery conditions.

The schematic demonstrates how the power circuit is designed to deliver a regulated +3.3V supply to the wearable sensor module 1, supporting the operation of the onboard electronics, sensors, and wireless communication components. The integration of these elements ensures efficient power management, extended battery life, and safe operation of the wearable sensor module 1 during continuous rehabilitation monitoring.

FIG. 10 illustrates the printed circuit board (PCB) layout for the wearable sensor module 1, highlighting the arrangement and integration of significant electronic components. The battery B1 is positioned to provide power to the entire module, ensuring reliable operation during extended use. The BLE Radio U2 is included to enable wireless communication between the wearable sensor module 1 and the companion software application on an external electronic device, supporting real-time data transmission and feedback.

The microcontroller U1 is centrally located on the PCB and is responsible for processing sensor data, executing signal processing algorithms, and managing communication protocols. The switch S1 provides user-initiated activation and control of the device, while the connector J1 facilitates external connections for charging or data transfer as required.

A series of capacitors, including C1, C2, C3, C4, C5, C16, and C17, are distributed across the PCB to stabilize voltage levels, filter noise, and ensure smooth operation of the electronic circuits. The resistors R5 and R8 are included to set appropriate electrical parameters for the circuit, contributing to the reliable performance of the module.

The PCB layout in FIG. 10 demonstrates a compact and efficient design, integrating the battery B1, BLE Radio U2, microcontroller U1, switch S1, connector J1, resistors R5 and R8, and multiple capacitors to support the advanced sensing, processing, and wireless communication functions required for effective rehabilitation monitoring and guidance.

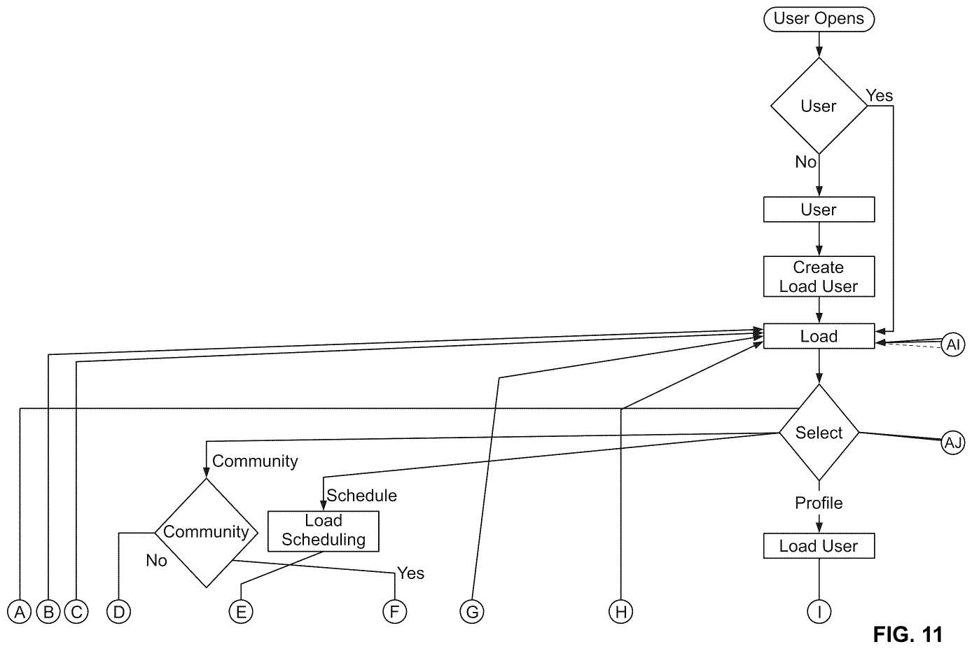

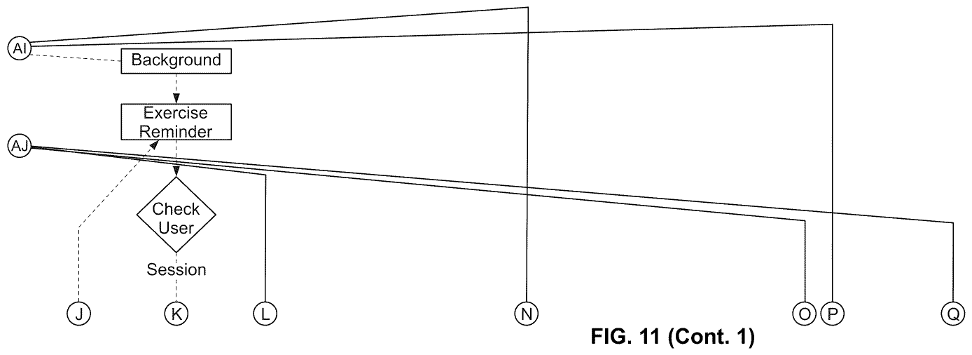

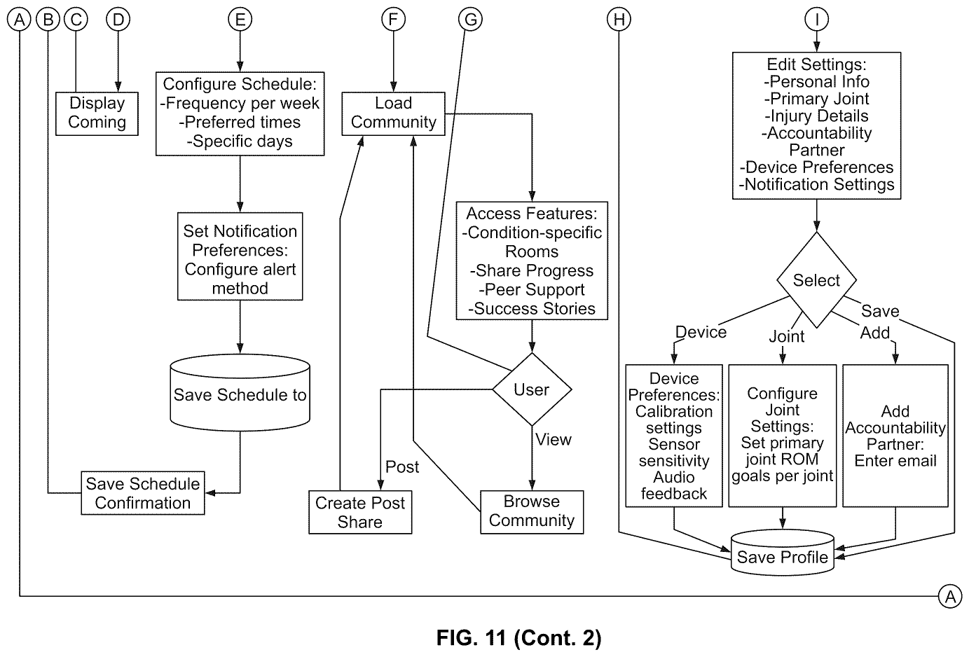

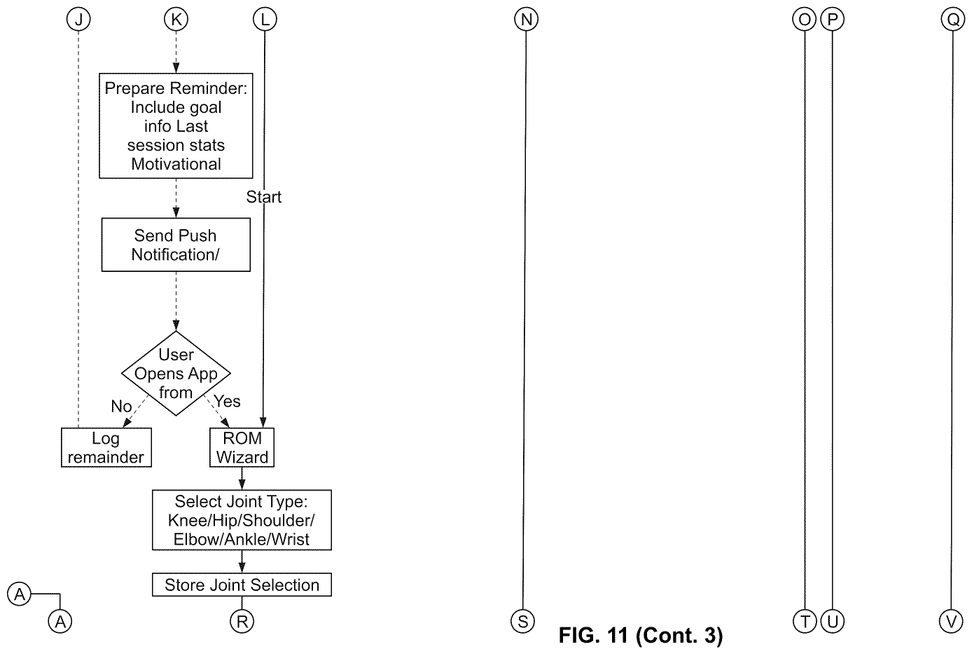

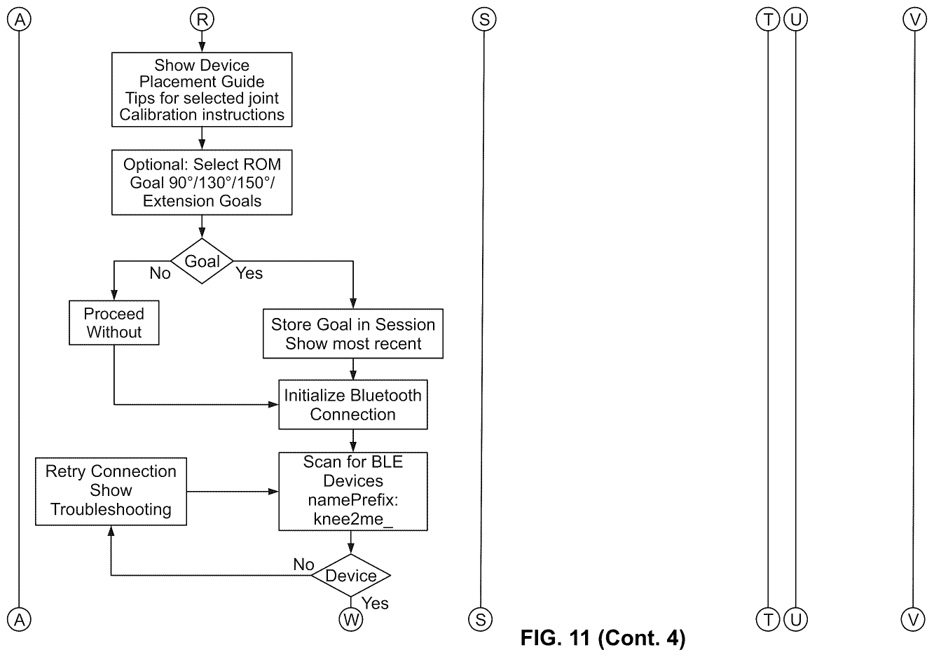

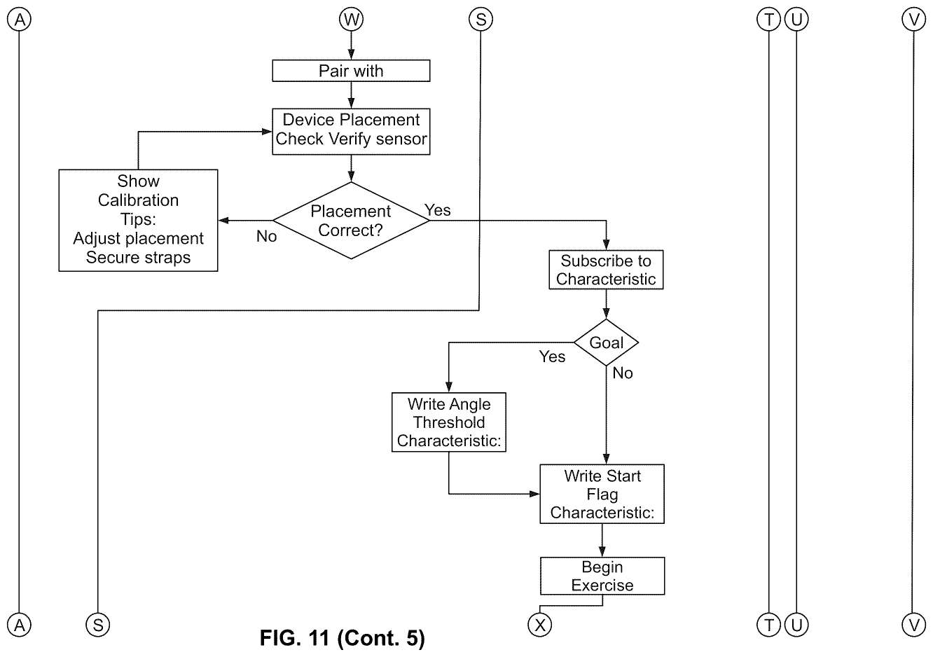

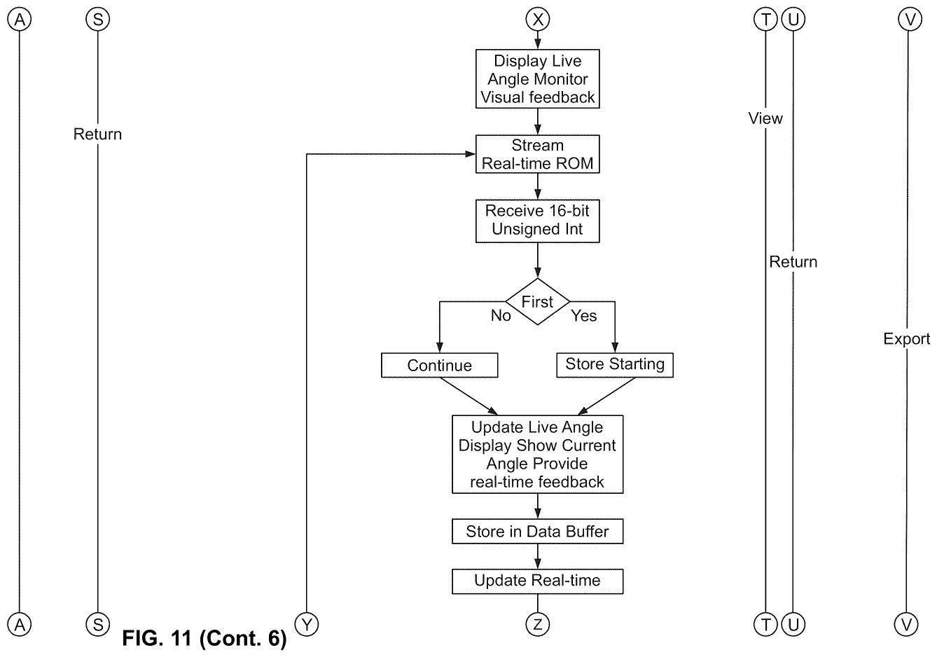

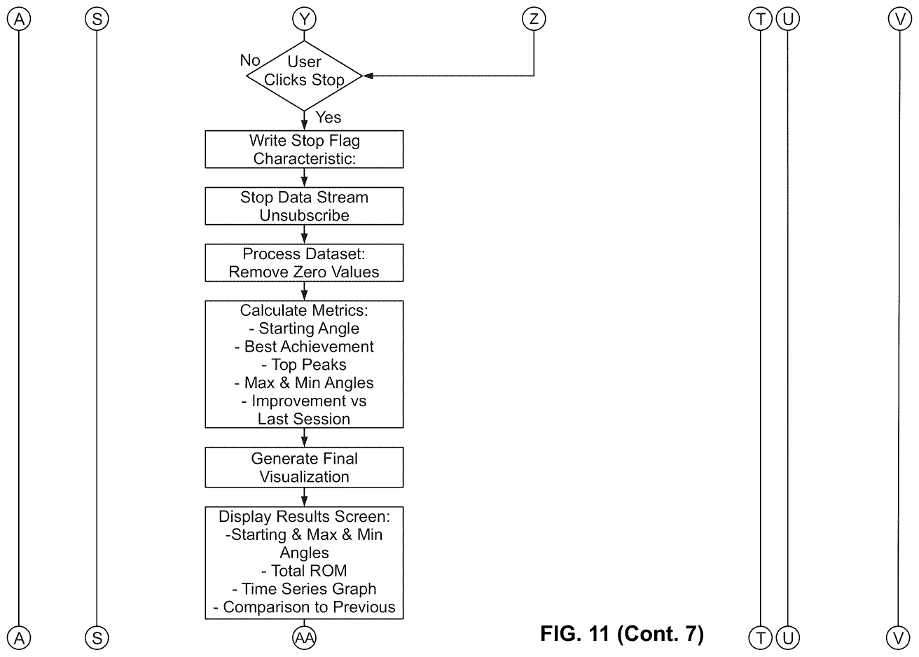

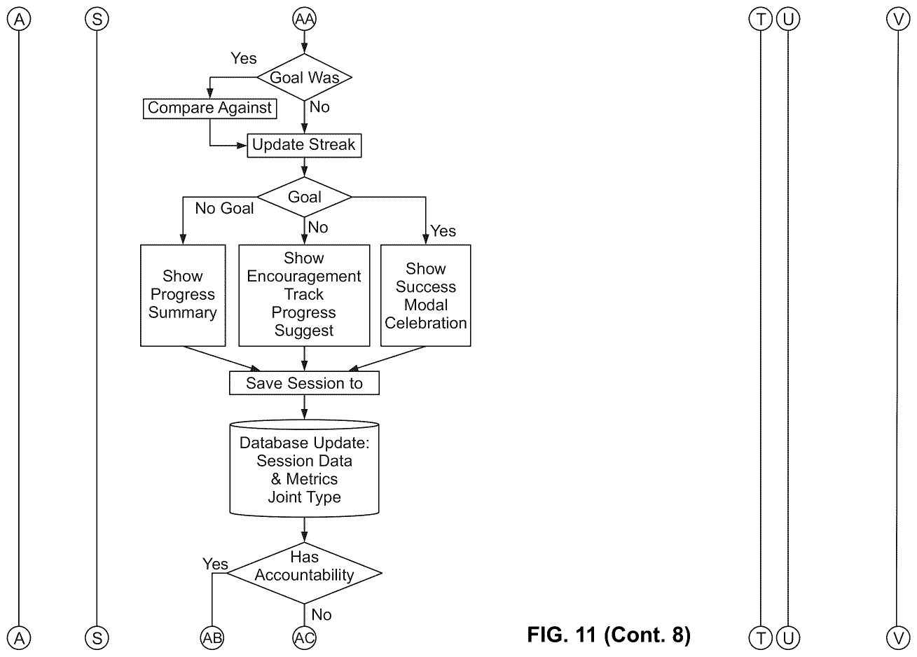

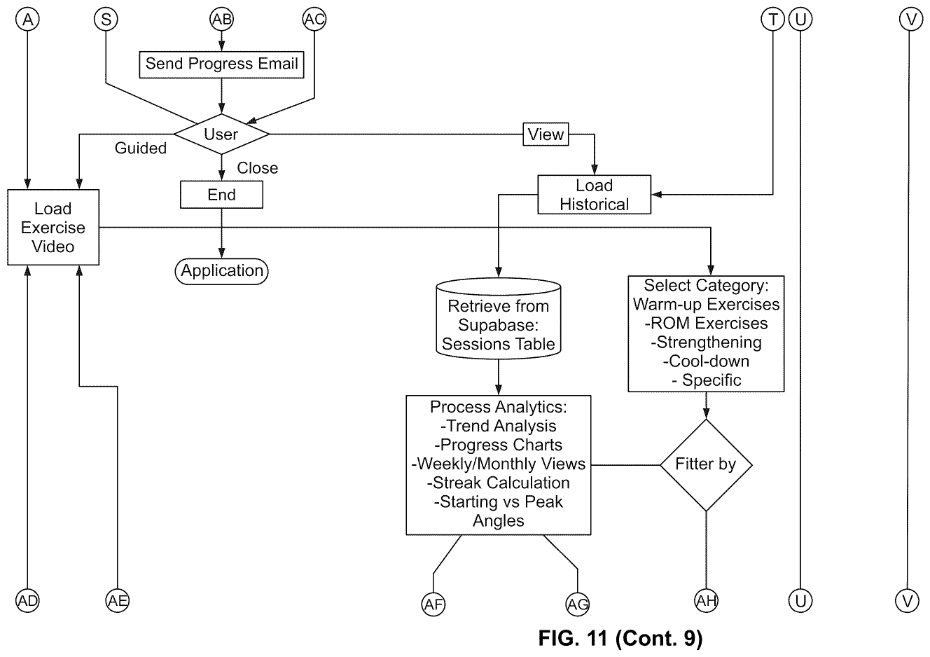

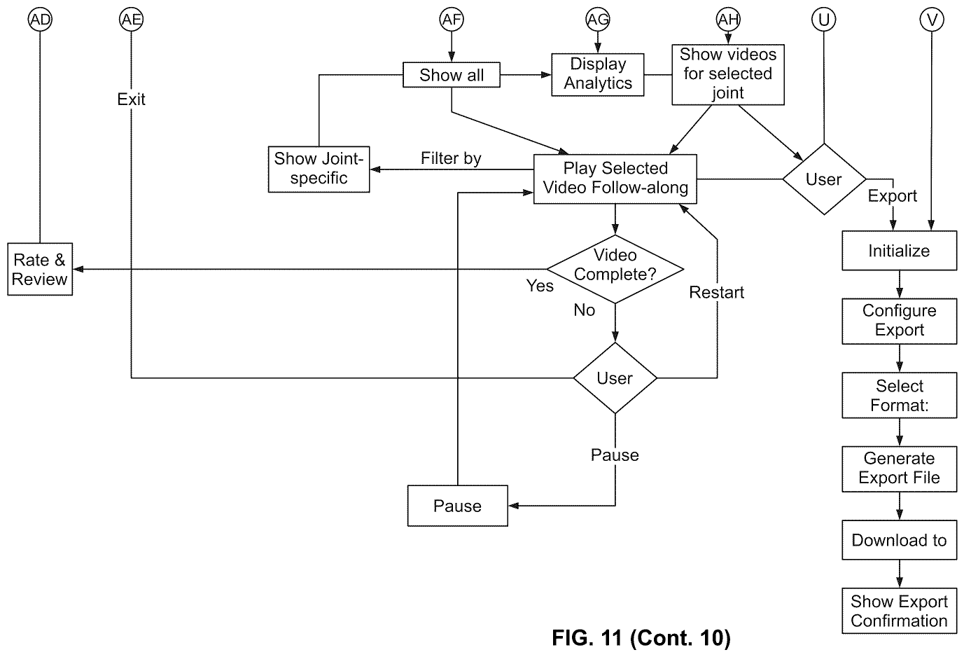

FIG. 11 and the associated continuations collectively illustrate a flowchart for one embodiment of a system for monitoring and guiding rehabilitation exercises using the wearable sensor module 1 and companion software application. The flowchart details the process of user interaction, device initialization, exercise session management, real-time monitoring, data processing, and progress tracking.

In FIG. 11, the process begins when a user opens the companion software application. The system checks if the user is already registered; if not, the user is prompted to create and load a user profile. The user can then select from various options, including accessing their profile, configuring scheduling preferences, or engaging with the community features. The scheduling module allows the user to set exercise frequency, preferred times, and notification preferences, which are saved for future sessions. The community module provides access to condition-specific information, peer support, and the ability to share progress or success stories.

The flow continues with the preparation and delivery of exercise reminders, which include goal information, past session statistics, and motivational content. When the user opens the application in response to a reminder, they are guided through a ROM (range of motion) wizard to select the joint type (such as knee, hip, shoulder, elbow, ankle, or wrist) if more than one joint type is available, and store their selection for the session.

Device initialization is supported by a placement guide, execution tips, and calibration instructions. The user may optionally select a ROM goal, which is stored for the session. The system then initializes a Bluetooth connection, scans for the wearable sensor module 1, and provides troubleshooting if the device is not detected. Once paired, the system verifies device placement and guides the user through calibration and securing the straps. Upon successful placement, the system subscribes to the sensor's data stream, writes angle threshold and start flag characteristics, and begins the exercise session.

During the exercise, the system displays a live angle monitor and provides real-time visual feedback. The wearable sensor module 1 streams real-time ROM data, which is received and processed by the companion software application. The application updates the live angle display, stores data in a buffer, and continuously provides feedback to the user.

When the user clicks stop, the system writes a stop flag characteristic, unsubscribes from the data stream, and processes the dataset by removing zero values. The application calculates important metrics such as starting angle, notable achievement, maximum and minimum angles, and improvements from the last session. Final visualizations are generated, and the results screen displays comprehensive performance data, including total ROM and time graphs with comparisons to previous sessions.

The system then compares the results against the user's goals, updates streak counters, and provides encouragement or celebratory feedback as appropriate. Session data and metrics are saved to the database, and if the user has an accountability partner, progress updates are shared accordingly.

Additional features include loading exercise videos, sending progress emails, viewing historical session data, and processing analytics such as trend analysis, progress charts, weekly ROM reviews, streak calculations, and standing peak angles. The user can filter exercises by category, such as warm-up, ROM, strengthening, or cool-down, and access detailed performance analytics to support ongoing rehabilitation and adherence.

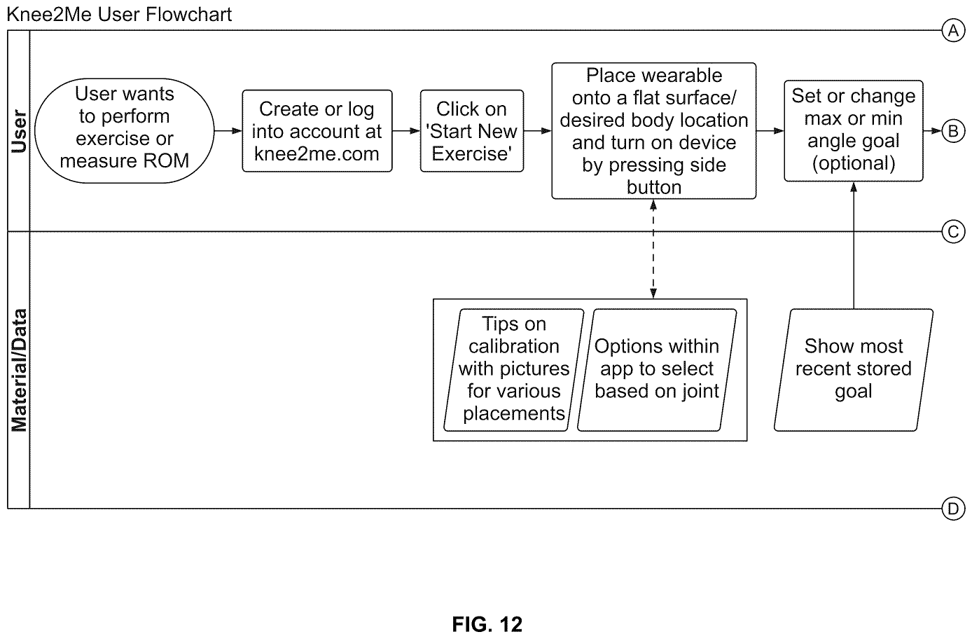

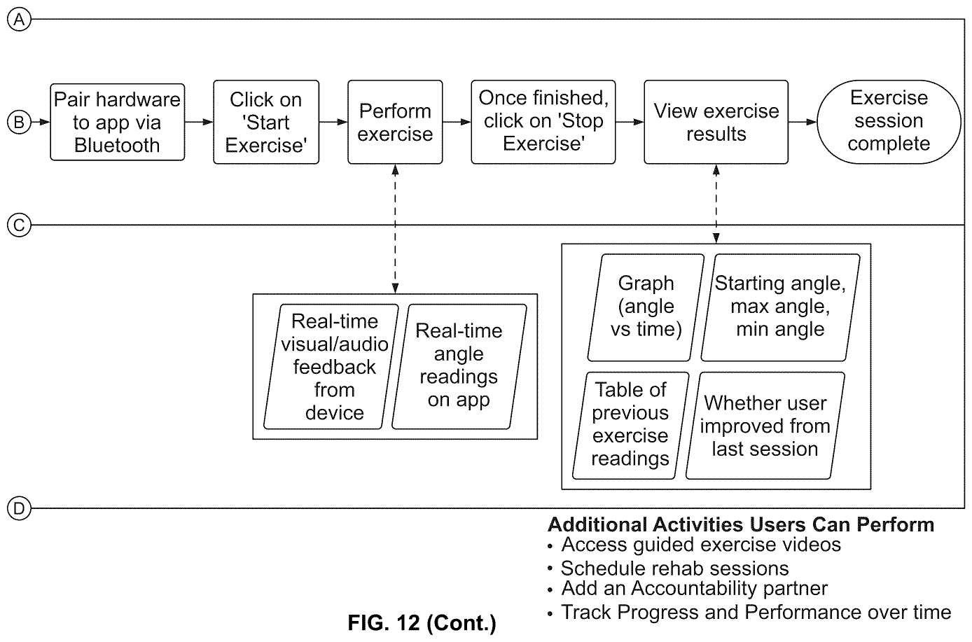

FIG. 12 illustrates a user flowchart for one embodiment of a system for monitoring and guiding rehabilitation exercises using the wearable sensor module 1 and the companion software application. The flowchart details the sequence of steps a user follows to initiate and complete an exercise session, as well as the associated features available within the system.

The process begins when a user wants to perform an exercise or measure range of motion (ROM). The user creates or logs into an account at knee2me. com. After authentication, the user clicks on ‘Start New Exercise’ to begin a session. The user is then prompted to place the wearable sensor module 1 onto a flat surface or the desired body location and turn on the device by pressing the side button. In another embodiment calibration happens automatically so the user won't need to start by placing object on flat surface or desired body part.

The user may optionally set or change the axis of rotation and configure maximum or minimum angle goals for the session. The system displays the most recent stored goal for reference. The application provides tips on calibration with pictures for various placements and offers options within the app to select the appropriate page based on the joint being exercised.

Once the device is ready, the user pairs the hardware to the app via Bluetooth and clicks on ‘Start Exercise.’ During the exercise, the user performs the prescribed movements while the system provides real-time visual and/or audio feedback from the device and displays real-time angle readings on the app. The application also generates a graph of angle versus time, displays starting, maximum, and minimum angles, and presents a table of previous exercise readings. The system indicates whether the user has improved from the last session.

Upon completion, the user clicks on ‘Stop Exercise’ and views the exercise results. The session is then marked as complete. Additional activities available to the user include accessing guided exercise videos, scheduling rehabilitation sessions, adding an accountability partner, and tracking progress and performance over time.

Additionally, the software may track progress by logging exercise adherence, improvements in range of motion, and recovery milestones on a day-by-day basis. To further support adherence, the application may include features such as push notifications for exercise reminders, an accountability partner feature for social support, and streak tracking to motivate users through visual rewards for consecutive exercise completion. Data visualization tools provide graphical summaries of range of motion over time, adherence trends, and feedback reports, which can be shared with care providers through cloud syncing. The application also supports offline data logging, ensuring continuous tracking even during temporary disconnections.

The companion software application, which is a part of the system, is designed to enhance the user experience by providing comprehensive support and feedback during rehabilitation exercises. This software is compatible with both mobile and desktop platforms, ensuring accessibility and convenience for users. It offers guided exercise protocols prescribed by physiotherapists, complete with instructional video demonstrations to ensure correct exercise execution. The application delivers real-time visual and auditory feedback on movement quality and range of motion, allowing users to make immediate adjustments to their technique.

FIG. 11 illustrates a possible flow diagram for the embodiment of FIG. 1 in communication with the companion software application.

As indicated above FIG. 12 is a User Flow Diagram illustrating one method of using the invention of the present disclosure. First after strapping the wearable sensor module around the joint such as the knee, the wearable sensor module is powered on and paired with an external electronic device having access to the companion software app. In the embodiment illustrated the pairing is via BLE but as noted above other protocols are possible. The patient signs in to the companion software app on the external electronic device. The patient selects the rehabilitation exercise and ROM target from their personalized rehabilitation exercises as prescribed by their therapist. The patient then performs the selected exercise wearing the sensor module. The sensor module tracks joint angles and range of motion. Sensor data is transmitted to the companion software app by way of the external electronic device and data analysis by the companion software app for real time feedback on the patient's progress is re-transmitted back to the sensor module. The onboard processing unit receives the data analysis and causes the appropriate visual and or audio indicator on the sensor module to provide real time feedback to the patient. The companion software app and external electronic device can provide the results of the patient's progress based on the sensor data collected, stored and can generate tables and charts etc. and these can be shared with the patient's therapist. The patient's streak of number of consecutive times the rehabilitation exercises are performed is updated.

The companion software app can also be programmed to permit access to guided exercise videos, schedule rehab sessions, add an accountability partner and track progress and performance over time. Streaks reward mechanisms may be provided by way of badges etc.

Social Support Module: A community engagement feature may be integrated within the companion software application, comprising condition-specific communication channels that enable peer-to-peer interaction among users undergoing similar rehabilitation protocols, thereby fostering accountability, shared learning, and sustained motivation through collective experience sharing and progress celebration.

Data Export Module: The companion software application includes functionality enabling users to extract and export their complete exercise performance data, including historical range-of-motion measurements, adherence statistics, progress trajectories, and graphical summaries in machine-readable and human-readable formats, facilitating data portability, provider communication, and integration with external health record systems.

Goal-Setting & Real-Time Comparison: Allows users to set custom ROM targets and receive real-time color-coded feedback (red/yellow/green) indicating whether current angles are below, approaching, or meeting their goal. Both on-device LED indicators and app-based displays provide immediate guidance, with post-exercise summaries comparing achieved ROM against targets and tracking progress over time.

Core Features of the Companion software app include:
Exercise Guidance: Guided rehab exercises prescribed by physiotherapists with instructional video demonstrations.
Real-Time Feedback: Live visual and auditory feedback on movement quality and range of motion.
Progress Tracking: Day-by-day logging of exercise adherence, ROM improvements, and recovery milestones.
Reminders & Scheduling: Push notifications to remind users to complete exercises. and to inform accountability partner when user has not completed exercise.
Accountability Partner Feature: Allows users to invite a friend, caregiver, or clinician to monitor their progress and encourage consistency.
Streak Tracking: Visual streak counters that reward users for completing exercises consecutively, reinforcing habit-building and motivation.
Data Visualization: Graphical summaries of ROM over time, adherence trends, and feedback reports.
Connectivity & Storage:
   Cloud syncing for backup and data sharing with care providers
   Offline mode for data logging during temporary disconnection

Having illustrated and described preferred embodiments of the invention and certain possible modifications thereto, it should be apparent to those of ordinary skill in the art that the invention permits of further modification in arrangement and detail and is not restricted to the embodiments illustrated.

It will be appreciated that the above description related to the preferred embodiments by way of example only. Many variations on the invention will be obvious to those knowledgeable in the field, and such obvious variations are within the scope of the invention as described and claimed, whether or not expressly described.

1. A rehabilitation monitoring system comprising:
a wearable sensor module configured to be secured around a user's body adjacent a joint being rehabilitated, the wearable sensor module including: an inertial measurement unit including range-of-motion sensors that jointly generate raw motion signals; an onboard processor configured to compute, in real time, joint-angle trajectories and exercise-performance metrics from the raw motion signals; and a wireless transceiver operable to transmit the computed exercise-performance metrics to an external electronic device and to receive configuration data from the external electronic device while the user is performing rehabilitation exercises; and a companion software application stored on the external electronic device and, when executed by the external electronic device, configured to: receive the exercise-performance metrics transmitted by the wearable sensor module; generate simultaneous visual and/or auditory feedback that guides the user toward correct movement in real time; and transmit the configuration data back to the wearable sensor module in accordance with detected user-performance trends; wherein the wearable sensor module and the companion software application communicate bidirectionally during performance of the rehabilitation exercises.

2. The rehabilitation monitoring system of claim 1 wherein the range-of-motion sensors comprise a gyroscope and an accelerometer that jointly generate raw motion signals to capture angular displacement of a joint.

3. The rehabilitation monitoring system of claim 1, wherein the companion software application stored on the external electronic device is configured to record each completed exercise repetition together with an adherence time-stamp.

4. The rehabilitation monitoring system of claim 1, wherein the wearable sensor module further comprises a selectively activatable magnetometer whose output is combined with the inertial measurement unit to enhance joint-angle accuracy when magnetic disturbance is below a stored threshold.

5. The rehabilitation monitoring system of claim 1, wherein the wearable sensor module further comprises a rechargeable battery, a USB-C charging port permanently integrated in the module housing, and an LED charge/status indicator.

6. The rehabilitation monitoring system of claim 1, wherein the wearable sensor module is retained by an adjustable elastic strap whose skin-facing surface comprises breathable fabric to permit multi-hour continuous wear without skin irritation.

7. The rehabilitation monitoring system of claim 1, wherein the wearable sensor module further comprises an outward-facing LED array controlled by the onboard processor to emit color-coded light patterns representative of current joint-angle magnitude so that the user receives direct visual feedback without reference to the external electronic device.

8. The rehabilitation monitoring system of claim 1, wherein the onboard processor is further configured to:
detect peak range-of-motion values for each exercise repetition; and
transmit only the detected peak values together with exercise-type metadata to the external electronic device.

9. The rehabilitation monitoring system of claim 1, wherein the onboard processor is further configured to apply at least one of Savitzky-Golay filtering, adaptive moving-average filtering, or Kalman filtering to the raw motion signals.

10. The rehabilitation monitoring system of claim 2, wherein a second sensor unit is provided configured to be secured around a user's body adjacent a joint being rehabilitated, the second sensor unit including an inertial measurement unit wherein outputs from the second sensor unit are used in conjunction with the wearable sensor module to provide a reference orientation for x, y, and z axes from which the range of motion is calculated by the wearable sensor module.

11. A computer-implemented method for guiding a user through a knee physiotherapy session without clinical supervision, the method comprising:
securing the wearable sensor module of claim 1 to the user's body adjacent the joint being rehabilitated;
calculating, on the wearable sensor module, joint-angle trajectories and exercise-performance metrics in real time;
wirelessly transmitting the exercise-performance metrics to a mobile computing device executing the companion software application;
comparing, in real time, the metrics with a stored target trajectory;
outputting an immediate corrective prompt selected from sound, vibration, light, or on-screen highlight whenever the comparison indicates deviation from the target trajectory.

12. The computer-implemented method of claim 11 comprising the further steps of upon completion of a set, recording a time-stamped entry that includes repetition count, maximum flexion angle or extension reached, and a movement-quality score.

13. The computer-implemented method of claim 12, further comprising automatically synchronizing the time-stamped entry to a cloud account accessible by an authorized accountability partner when network connectivity is present, and storing the entry locally for later synchronization when connectivity is absent.

14. The computer-implemented method of claim 11, further comprising issuing a reminder notification to a user or a user's and accountability partner when a scheduled exercise session is missed.

15. A gamified rehabilitation software application executable on a portable electronic device, the application being configured to:
receive, via a wireless interface, exercise-performance metrics originating from a wearable knee-rehabilitation sensor module; share aggregate performance metrics with at least one designated accountability partner through a network connection; unlock guided exercise videos; and visually represent user progress on an interactive chart that is updated after each exercise session.

16. The gamified rehabilitation software application of claim 15, further configured to export stored performance metrics as both human-readable PDF reports and machine-readable FHIR-compliant records for sharing with clinicians and integration into external electronic health-record systems.
